(19) United States
(12) Patent Application Publication
(10) Pub. No.: US 2026/0137914 A1
(43) Pub. Date: May 21, 2026

(54) WIRELESS INTELLIGENTLY CONTROLLED DRUG-RELEASING INTRAVESICAL FLOATING DRUG DELIVERY DEVICE AND SYSTEM

(71) Applicants: Zhihui Zou, Hefei (CN); THE FIRST AFFILIATED HOSPITAL OF ANHUI MEDICAL UNIVERSITY, Hefei (CN)

(72) Inventors: Zhihui Zou, Hefei (CN); Siyuan Xia, Hefei (CN); Jiaqing Zou, Hefei (CN); Shuchen Liu, Hefei (CN); Kaixuan Zhang, Hefei (CN); Han Yang, Hefei (CN); Zhengyang Wu, Hefei (CN); Qi Zhang, Hefei (CN); Zicheng Li, Hefei (CN); Jiaying Song, Hefei (CN); Lele Qin, Hefei (CN); Yongtao Hu, Hefei (CN); Yu He, Hefei (CN); Keke Cai, Hefei (CN); Ligang Zhang, Hefei (CN); Zongyao Hao, Hefei (CN); Chaozhao Liang, Hefei (CN)

(21) Appl. No.: 19/389,109

(22) Filed: Nov. 14, 2025

(30) Foreign Application Priority Data
Nov. 15, 2024 (CN) 2024116361022

(51) Int. Cl. A61M 31/00

(52) U.S. Cl. CPC A61M 31/002; A61M 2205/3523; A61M 2210/1085

(57) ABSTRACT

A wireless intelligently controlled drug-releasing intravesical floating drug delivery device and system, the device includes a housing, a drive module, a control module, and a power supply module. The housing is configured to enter the bladder via the urethra and float within the intravesical urine, the housing is internally provided with at least one drug chamber for containing drug and a gas chamber for providing buoyancy. The drive module is disposed within the housing and configured to cause drug to be discharged from the drug chamber to the exterior of the housing in a timed and/or quantified manner. The control module is configured to control the operation of the drive module. The power supply module is disposed within the housing and configured to supply power to the drive module. The system reduces the need for repeated, long-term, and invasive catheterization procedures during bladder perfusion therapy.

### FIELD OF THE INVENTION

The present invention relates to the technical field of medical devices, and more particularly to a wireless intelligently controlled drug-releasing intravesical floating drug delivery device and system, including a intravesical floating drug delivery device, a placement rod, a intravesical floating drug delivery device component, and a control method for the device.

### BACKGROUND OF THE INVENTION

Bladder perfusion therapy is a traditional treatment for non-muscle-invasive urothelial carcinoma, serving as an adjuvant therapy after surgery. This method involves instilling drugs into the bladder, which kill remaining cancer cells through the drug's cytotoxicity or by stimulating the immune system's response to the tumor. This adjuvant treatment is suitable for patients who have undergone transurethral resection of bladder tumors (TURBT), aiming to prevent tumor recurrence.

The bladder, with its urine urine-storing cavity, provides an anatomical basis for implementing bladder perfusion therapy. Currently, bladder perfusion therapy requires inserting a catheter through the urethra into the bladder and slowly injecting the drug from outside the body. This necessitates repeated, long-term, and invasive catheterization. Such catheterization may be required monthly, even for over a year, making this repetitive procedure highly prone to postoperative complications such as infection, bleeding, and urethral stricture. It may even lead to failure of catheter insertion, resulting in an inability to persist with the bladder perfusion therapy. The occurrence of various postoperative complications causes significant physiological and psychological trauma to patients, leading to suspicious, pessimistic, and anxious negative psychological states.

### SUMMARY OF THE INVENTION

Therefore, the object of the present invention is to provide a wireless intelligently controlled drug-releasing intravesical floating drug delivery device and system, which addresses the issue of existing bladder perfusion therapy requiring repeated transurethral operations that cause great patient suffering. The system includes a intravesical floating drug delivery device, a placement rod, a intravesical floating drug delivery device component, and a control method for the device.

The present invention provides a wireless intelligently controlled drug-releasing intravesical floating drug delivery device, the device includes a housing, a drive module, a control module, and a power supply module. The housing is configured to enter the bladder via the urethra and float within the intravesical urine, and internally provided with at least one drug chamber for containing drug and a gas chamber for providing buoyancy to float the entire intravesical floating drug delivery device within the intravesical urine. The drive module is disposed within the housing, and configured to cause drug to be discharged from the drug chamber to the exterior of the housing in a timed and/or quantified manner. The control module is configured to control the operation of the drive module. The power supply module is disposed within the housing, and configured to supply power to the drive module.

In the device of the present invention, the housing is connected with a plurality of partition plates; the plurality of partition plates partition the interior of the housing into a plurality of drug chambers; the cross-section of each of the drug chambers is fan-shaped and all cross-sections are concentrically arranged; the housing is provided with first through holes corresponding one-to-one with the plurality of drug chambers, the first through holes are configured for the ingress and egress of corresponding drug.

In the device of the present invention, a central tube is disposed at the center of the plurality of fan-shaped chambers; the drive module includes:
a motor configured to be controlled by the control module and rotate by a set angle;
a transmission rod rotatably disposed inside the central tube; one end of the transmission rod is connected to an output shaft of the motor; and
a baffle plate connected to the other end of the transmission rod, wherein the baffle plate is rotatably connected to the housing and configured to seal the side of the housing provided with the first through holes; the baffle plate is configured to close or open the first through holes upon rotation.

In the device of the present invention, the drive module includes:
a first electromagnet disposed at one end of the drug chamber; the first electromagnet is configured to be controlled by the control module to generate a magnetic force;
a permanent magnet sealed and slidably disposed along an inner wall of the drug chamber; the magnetic force of the permanent magnet is repulsive to the magnetic force of the first electromagnet, and a portion of the drug chamber on a side of the permanent magnet away from the first electromagnet is a drug reservoir; and
a check valve; the housing is provided with a first through hole in communication with the drug reservoir, the check valve is disposed within the first through hole, and an opening direction of the check valve is from the interior to the exterior of the housing.

In the device of the present invention, two adjacent sensing electrodes are disposed within each drug chamber; the two sensing electrodes are configured to generate a conduction signal after contacting the urine or drug within the bladder, the conduction signal is transmitted to an external device via the control module.

In the device of the present invention, the control module includes:
a storage unit configured to store preset control programs; and
a processing unit configured to control the operation of the drive module according to the control programs.

In the device of the present invention, the control module includes:
a wireless unit configured to remotely receive signals from outside the housing; and
a processing unit configured to control the operation of the drive module based on the signals.

In the device of the present invention, the device further includes:
a camera module disposed within the housing; the camera module is configured to capture images of the bladder inner wall through the housing and transmit the captured video to the exterior via the wireless unit;
a light source disposed within the housing; the light source is configured to illuminate the shooting direction of the camera module through the housing; the power supply module is configured to supply power to the camera module and the light source.

In the device of the present invention, the gas chamber is configured as a semi-enclosed structure located at the head end and the side surfaces of the housing.

In the device of the present invention, the housing has a diameter not greater than 8 mm and an overall length not greater than 4 cm, and the outer surface of the housing is coated with an antibacterial, hydrophilic, anti-adhesion coating

In the device of the present invention, the check valve includes a rigid film provided with an incision, and when subjected to pressure from the drug within each drug chamber, the rigid film opens to allow unidirectional outflow of the drug.

The present invention also provides a placement rod for a intravesical floating drug delivery device, including a rod body and an engagement structure at one end of the rod body, wherein the engagement structure is configured to detachably engage with the device according to the above intravesical floating drug delivery device.

In the placement rod of the present invention, the housing is externally provided with a docking slot, and a magnet is connected within the docking slot; the engagement structure includes a docking head matching the docking slot and a second electromagnet disposed on the docking head, the second electromagnet is configured to attract the magnet when energized.

In the placement rod of the present invention, the engagement structure includes an operating handle and a clamp for securing the intravesical floating drug delivery device; the clamp is disposed at one end of the rod body, the operating handle is disposed at the other end of the rod body; the operating handle is configured to drive the opening and closing of the clamp.

In the placement rod of the present invention, the placement rod is integrated with an endoscope system, the endoscope system is an optical or electronic system.

In the placement rod of the present invention, the placement rod is provided with an inlet pipeline and an outlet pipeline; one end opening of the inlet pipeline and outlet pipeline is located at the end of the placement rod, and the other end is connected to an external water supply device for injecting and discharging urine into and from the bladder.

The present invention also provides a intravesical floating drug delivery device component, including the intravesical floating drug delivery device 1 and the placement rod.

The present invention also provides a control method for a wireless intelligently controlled drug-releasing intravesical floating drug delivery device, including the following step:
remotely transmitting a control signal to a control module of the intravesical floating drug delivery device to adjust a drug release timing or a drug release amount.

The present invention also provides a control method for a wireless intelligently controlled drug-releasing intravesical floating drug delivery device having a control module and a drive module, including the following step:
starting the drive module when a preset start time on the control module arrives; and
after the drive module is started, controlling the periodic operation of the drive module according to a preset cycle period, wherein the cycle period comprises an operating time t1 and an interval time t2; stopping the drive module when the operating time t1 is reached; and starting the drive module when the interval time t2 is reached.

In the control method of the present invention, further includes the following step:
monitoring the conduction signal generated by the sensing electrodes and confirming the drug release status based on the conduction signal.

To solve the above problem, the present invention provides that once the intravesical floating drug delivery device is inserted into the bladder, the operator can perform multiple, timed, and quantified intravesical drug administration as needed. After the treatment is completed, the intravesical floating drug delivery device is finally removed from the bladder. The entire treatment process involves only two catheterization operations. Compared to existing bladder perfusion therapy, this avoids repeated, long-term, and invasive catheterization, thereby significantly reducing the immense suffering inflicted on patients by bladder perfusion therapy.

Existing bladder perfusion therapy requires operation by professional medical staff and specialized equipment in a hospital setting. The disposable, insertable drug-releasing intravesical floating drug delivery device designed in the present invention only requires hospital visits for the insertion and removal procedures. The remaining drug administration process can be performed anywhere, anytime, or on schedule by the patient themselves, thus effectively conserving medical resources and reducing treatment costs for patients.

### BRIEF DESCRIPTION OF THE DRAWINGS

The above objects and advantages of the present invention will become more readily apparent to those ordinarily skilled in the art after reviewing the following detailed description and accompanying drawings, in which:

FIG. 1 is a structural schematic diagram of the intravesical floating drug delivery device according to Embodiment 1.

FIG. 2 is a cross-sectional view taken along line A-A in FIG. 1.

FIG. 3 is a perspective structural view of the housing according to Embodiment 1.

FIG. 4 is a structural view of the interior of the housing according to Embodiment 2.

FIG. 5 is a structural schematic diagram of the intravesical floating drug delivery device according to Embodiment 3.

FIG. 6 is a cross-sectional view taken along line B-B in FIG. 5.

FIG. 7 is a structural schematic diagram of the intravesical floating drug delivery device according to Embodiment 4.

FIG. 8 is a cross-sectional view taken along line C-C in FIG. 7.

FIG. 9 is a structural schematic diagram of the intravesical floating drug delivery device according to Embodiment 5.

FIG. 10 is a structural schematic diagram of the intravesical floating drug delivery device according to Embodiment 6.

FIG. 11 is a structural schematic diagram of the placement rod according to Embodiment 8.

FIG. 12 is a partial structural view showing the engagement between the rod body and the housing according to Embodiment 8.

FIG. 13 is a partial structural view showing the engagement between the rod body and the housing according to Embodiment 9.

FIG. 14 is a partial structural view showing the engagement between the rod body and the housing according to Embodiment 10.

FIG. 15 is a structural schematic diagram of a placement rod integrated with an optical endoscope system according to Embodiment 11.

FIG. 16 is a structural schematic diagram of a placement rod integrated with an electronic endoscope system according to Embodiment 11.

FIG. 17 is a structural schematic diagram of a placement rod provided with inlet and outlet pipelines according to Embodiment 12.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

The present invention provides a wireless intelligently controlled drug-releasing intravesical floating drug delivery device, which only needs to be inserted into the bladder through the urethra once and then floated within the intravesical urine. The device includes a housing 1, a drive module, a control module 2, and a power supply module 3.

The housing 1 is configured to enter the bladder via the urethra and float within the intravesical urine. The housing is internally provided with at least one drug chamber 4 for containing drug and a gas chamber for providing buoyancy to float the entire intravesical floating drug delivery device within the intravesical urine. The drive module is disposed within the housing 1 and configured to cause drug to be discharged from the drug chamber 4 to the exterior of the housing 1 in a timed and/or quantified manner. The control module 2 is disposed within the housing 1 and configured to control the operation of the drive module. The power supply module 3 is disposed within the housing 1, and configured to supply power to the drive module.

Design a disposable drug-releasing intravesical floating drug delivery device for bladder insertion through the above structure. Once the intravesical floating drug delivery device is inserted into the bladder, the operator can perform multiple, timed, and quantified intravesical drug administration as needed. This avoids repeated, long-term, and invasive catheterization, thereby significantly reducing the immense suffering inflicted on patients by bladder perfusion therapy. Specifically, based on the idea of avoiding repeated catheterization operations, the specific design structure of the intravesical floating drug delivery device can have various forms, please refer to the following embodiments.

### Embodiment 1

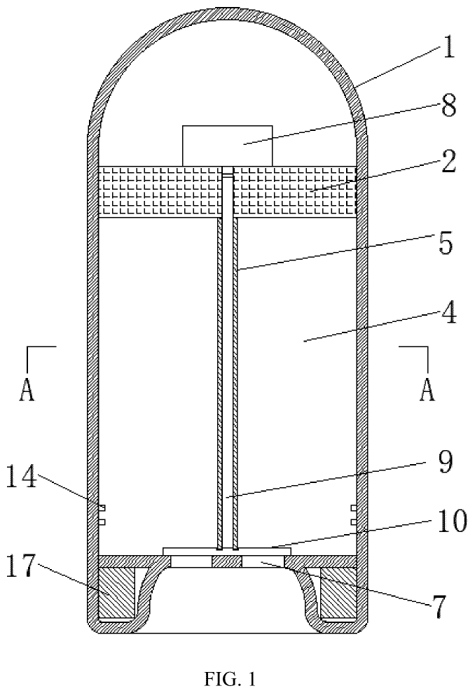

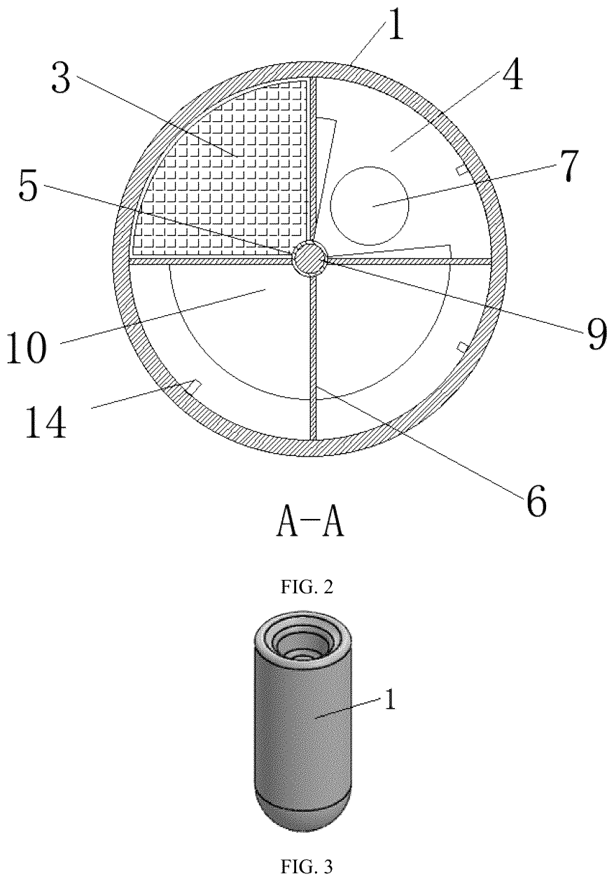

Please refer to FIGS. 1 and 2, a drug-releasing intravesical floating drug delivery device is shown as an embodiment. The device includes a housing 1, a drive module, a control module 2, and a power supply module 3. To reduce the pain and injury during insertion into the urethra, the overall shape of the housing 1 approximates a cylinder, as shown in FIG. 3, with a diameter not greater than 8 mm and an overall length not greater than 4 cm (excessive length makes insertion into the urethra difficult and can easily irritate the bladder wall). One end of the housing 1 is designed as an arc surface. This end can be regarded as the head end, and the other end as the tail end. When the housing 1 passes through the urethra, the head end leads. The outer surface of the housing 1 can be coated overall with an antibacterial, hydrophilic, anti-adhesion coating to reduce irritation to the urethra during insertion.

The drug chamber 4 and the gas chamber inside the housing 1 are two independent cavities. The gas chamber can be filled with air or other inert gases to provide buoyancy for the entire floating device. The size of the gas chamber (i.e., the buoyancy) is designed such that the entire floating device floats within the bladder urine or on the surface of the urine, preventing the housing 1 from contacting the sensitive trigone area at the bottom of the bladder, thereby avoiding patient discomfort. At least one drug chamber 4 is provided. The drug chamber 4 and the gas chamber can be separated by the control module 2.

This control module 2 can be an integrated circuit including a substrate. The integrated circuit includes a wireless unit and a processing unit. The wireless unit is configured to receive control signals from external devices (such as tablets, computers, or mobile phone apps), which include start commands like activation time, interval time, and motor rotation angle. The processing unit is configured to control the operation of the drive module based on the signals. Specifically, the processing unit can read the start commands and control the drive module to perform corresponding actions, allowing external devices to remotely control the drug release process of the intravesical floating drug delivery device. This enables the user or doctor to control the dosage and timing of drug administration in real-time based on the progression of the condition or the presence of adverse reactions.

The substrate is encapsulated in a waterproof sealing material or a waterproof shell and fixed inside the housing 1, directly serving as the separation layer between the drug chamber 4 and the gas chamber. The gas chamber is located above the substrate, and the drug chamber 4 is located below the substrate, enhancing the compactness of the internal structure of the housing 1. The drug chamber 4 is divided into multiple independent cavities based on the number of doses or the amount of drug to be administered.

The housing 1 is connected with a plurality of partition plates 6. The plurality of partition plates 6 partition the interior of the housing 1 into a plurality of drug chambers 4. The cross-section of each of the drug chambers 4 is fan-shaped and all cross-sections are concentrically arranged. The housing 1 is provided with first through holes 7 corresponding one-to-one with the plurality of drug chambers 4, the first through holes 7 are configured for the ingress and egress of corresponding drug.

This embodiment uses four drug chambers 4 as an example. A central tube 5 is fixedly connected to the central bottom position of the control module 2. Four partition plates 6 are uniformly fixed around the circumference of the central tube 5. The top, bottom, and ends of the four partition plates 6 away from the central tube 5 are all connected to the inner wall of the housing 1. Thus, every two adjacent partition plates 6, the control module 2, and the housing 1 form an independent drug chamber 4. The four drug chambers 4 are of uniform size and specification, with a fan-shaped cross-section. Three of these chambers can independently contain powdered or urine drug, and the fourth chamber can contain the power supply module 3.

The power supply module 3 includes a rechargeable battery, which is fully charged before the intravesical floating drug delivery device is inserted into the bladder. Three first through holes 7 are provided at the tail end of the housing 1. The three first through holes 7 are located at the same virtual circle, meaning they are equidistant from the transmission rod 9. The three first through holes 7 correspond one-to-one and connect to the three drug chambers 4 containing drugs. It is worth mentioning that the first through holes 7 should be made as large as possible without obstructing other structures to facilitate the passage of drug or urine.

Each drug chamber 4 is also provided with two adjacent sensing electrodes 14. The two sensing electrodes 14 are distributed vertically and fixed on the inner wall of the housing 1. The two sensing electrodes 14 are electrically connected to the control module 2. When they come into contact with urine or urine urinedrug, a connected circuit is formed between the two sensing electrodes 14, generating a conduction signal.

The conduction signal is transmitted to the control module 2 and sent to an external device via the wireless unit for analysis: if the drug chamber 4 contains powdered drugs, when the drug is discharged, urine immediately flows into and fills the drug chamber 4, thereby generating a conduction signal, which also indicates that the drug in the chamber has been discharged. Monitoring the presence or change of the conduction signal tracks whether the drug has been successfully released, preventing treatment delays due to device failure to release the drug.

The drive module includes a motor 8, a transmission rod 9, and a baffle plate 10. The motor 8 can be fixed above the control module 2 (i.e., within the gas chamber) and is controlled by the control module 2 to rotate by a set angle. The motor 8 needs to be a micro-motor matching the size of the housing 1, such as the mature rotor motors used in mobile phones, with diameters controllable to around 3 mm. The output shaft of the motor 8 faces downward, and its end is connected and fixed to one end of the transmission rod 9.

The transmission rod 9 passes through the control module 2, extends into the central tube 5, and is rotatably connected to the central tube 5. The end of the transmission rod 9 away from the motor 8 is fixed to the horizontally oriented baffle plate 10. The baffle plate 10 is rotatably connected to the housing 1 and configured to seal the side of the housing 1 provided with the first through holes 7. The baffle plate 10 is configured to close or open the first through holes 7 upon rotation.

In this embodiment, the baffle plate 10 is sealably connected to the tail end of the housing 1. A portion of the partition plate 6 at this location can be cut away to prevent interference with the baffle plate 10, or the baffle plate 10 can be directly embedded in the side wall of the housing 1 tail end for rotation. The continuous rotation of the baffle plate 10 serves to close or open the first through holes 7.

In this embodiment, the baffle plate 10 is shaped like a circular sector. The transmission rod 9 is fixed at the center of the sector. The circular sector has a notch compared to a full circle. In the initial position, this notch is located below the power supply module 3, and at this time, the baffle plate 10 blocks the first through holes 7, keeping the drug chambers 4 containing drugs in a closed state. When the notch rotates to align with a first through hole 7, that first through hole 7 is opened. At this point, urine from the bladder enters the drug chamber 4 through the first through hole 7, dissolves the drug, and allows the drug molecules to diffuse into the bladder mucosa with the flow of urine. The baffle plate 10 covers the first through hole 7 to close it.

The arc angle α of the sector-shaped baffle plate 10 must satisfy: (270°)<α<(360°−β), where β is the angle between one of first through holes 7 and the tangent of the center of the baffle plate 10, ensuring that the baffle plate 10 can open each drug chamber 4 sequentially, not simultaneously. A protrusion is provided at the edge of the tail end of the housing 1, forming a docking slot. An annular magnet 17 is provided inside the docking slot. The annular magnet 17 can be magnetically attracted to external devices, facilitating the movement and operation of the entire intravesical floating drug delivery device.

### Embodiment 2

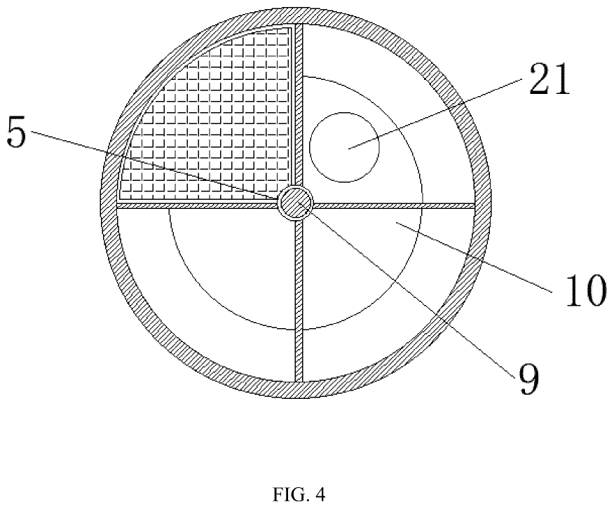

This embodiment differs from Embodiment 1 primarily in the structural design of the baffle plate 10; the rest of the structure is the same as in Embodiment 1. Please refer to FIG. 4. The baffle plate 10 in this embodiment is circular. A second through hole 21 is provided in the circular baffle plate 10. Since all the first through holes 7 lie on the same virtual circle, as long as the second through hole 21 can align with one of the first through holes 7, it can align with the others through rotation. When the second through hole 21 aligns with a first through hole 7, the drug chamber 4 communicates with the exterior of the housing 1, initiating drug release. When the second through hole 21 is misaligned with the first through holes 7, the baffle plate 10 closes the first through holes 7, sealing the drug chamber 4 from the exterior of the housing 1.

### Embodiment 3

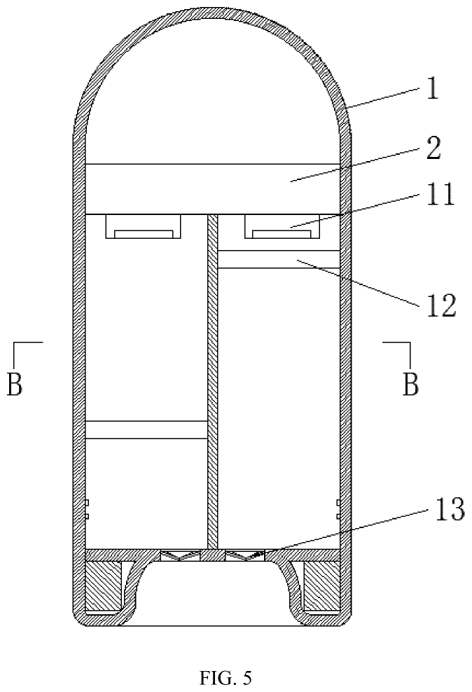

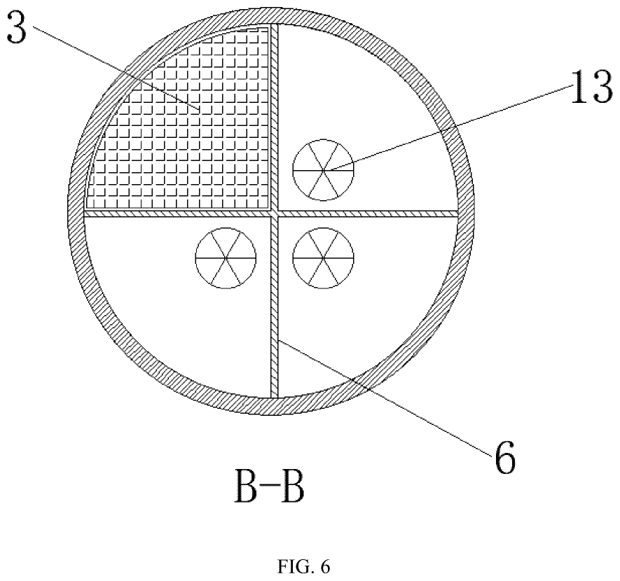

This embodiment differs from Embodiment 1 primarily in the structural design of the drive module; the rest of the structure is the same as in Embodiment 1. In this embodiment, each drug chamber 4 containing drugs is equipped with its own drive module, as shown in FIGS. 5 and 6, including a first electromagnet 11, a permanent magnet 12, and a check valve 13. The first electromagnet 11 is fixed at the bottom of the control module 2, electrically connected to the power supply module 3, and controlled by the control module 2 to generate a magnetic force through the switching of electrical power.

The permanent magnet 12 is sheet-like, oriented horizontally, and sealed and slidably disposed along the inner wall of the drug chamber 4. It is important to note that the magnetic force of the permanent magnet 12 is repulsive to the magnetic force of the first electromagnet 11. That is, when the first electromagnet 11 is energized and produces a magnetic force, it pushes the permanent magnet 12 downward. A portion of the drug chamber on a side of the permanent magnet away from the first electromagnet is a drug reservoir which in communication with the first through hole 7. In the initial state, the permanent magnet 12 is located in the upper region of the drug chamber 4. The space below the permanent magnet 12 within each drug chamber 4 is the drug reservoir, used for storing the drug.

The check valve 13 is installed within the first through hole 7, and its opening direction is from the inside to the outside of the drug chamber 4. That is, the opening direction of the check valve 13 is from the interior to the exterior of the housing 1. This check valve 13 can be a rigid film with a cross or star-shaped incision. Normally, when not under force, the film is gathered at the incision, remaining closed. When the permanent magnet 12 moves downward, it pushes the drug, causing the film to be subjected to pressure from the drug. The film then opens outward at the incision under force, forming a passage through which the drug enters the bladder.

Furthermore, the central part of the rigid film slightly protrudes outward from the drug chamber 4, making it less likely to be pushed open by external forces from outside the chamber, creating a one-way flow effect. In this embodiment, since the interior of the drug chamber 4 is emptied by the permanent magnet 12, the two sensing electrodes 14 can detect whether the urine drug has been discharged: if the drug chamber 4 contains a urine drug, when the drug is discharged, the conduction signal disappears, indicating that the drug in the chamber has been successfully discharged.

### Embodiment 4

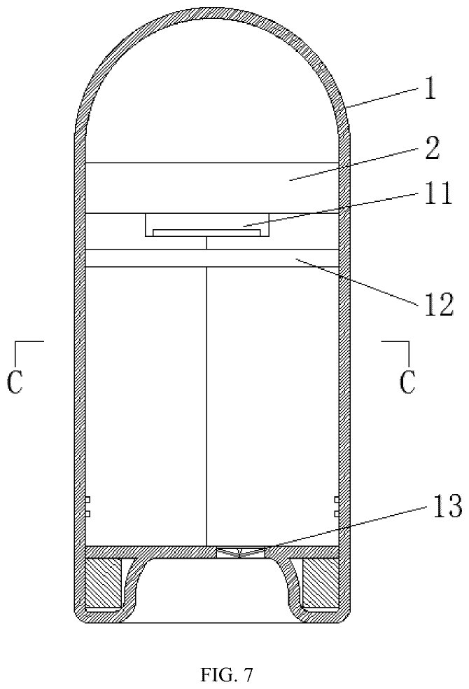

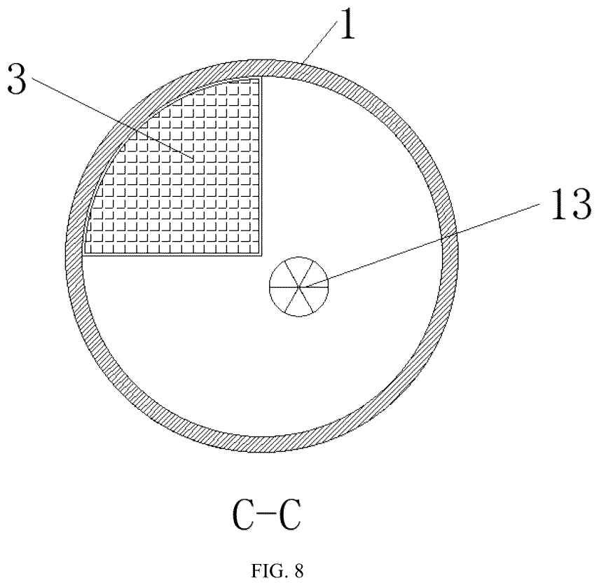

This embodiment uses the same drive module as in Embodiment 3 but differs in that it employs only one drug chamber 4. Accordingly, only one first through hole 7 needs to be provided; the rest of the structure is the same. The battery is still installed within each drug chamber 4. As shown in FIGS. 7 and 8, this embodiment has only one drug chamber 4. The first electromagnet 11 is similarly fixed at the bottom of the control module 2.

The permanent magnet 12 needs to match the cross-section of the drug chamber 4 excluding the battery space, sliding up and down within the remaining portion of the drug chamber 4. The downward movement of the permanent magnet 12 ultimately pushes the drug out through the check valve 13 into the bladder, making the entire drug chamber 4 structure and principle of action similar to a syringe. This is suitable for urine drugs and results in a more streamlined structure for the intravesical floating drug delivery device. The amount of drug released is controlled by the downward travel distance of the permanent magnet 12. This travel distance can be controlled based on the magnitude and duration of the repulsive force between the permanent magnet 12 and the first electromagnet 11, aiming to minimize the installation of other components.

### Embodiment 5

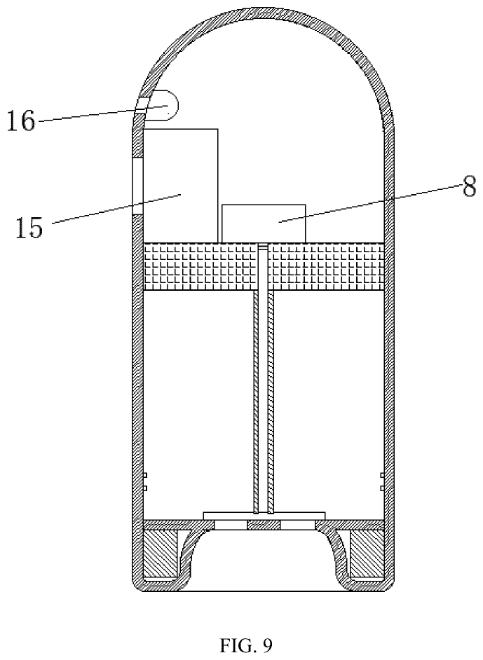

Compared to the previous embodiments, this embodiment expands the gas chamber portion and additionally includes a camera module 15 and a light source 16 within the gas chamber; the rest of the structural design is the same as in the previous embodiments. As shown in FIG. 9, the housing 1 is provided with an opening. The camera module 15 and the light source 16 are fixed to the control module 2 or the inner wall of the housing 1. The lens of the camera module 15 captures images of the bladder inner wall through the housing 1 by passing the opening. The light source 16 illuminates the shooting direction of the camera module 15 through the housing 1 by passing the opening. Both are powered by the power supply module 3. The camera module 15 is also connected to external devices via the wireless unit.

The control module 2 controls the activation and deactivation of the camera module 15 and light source 16 based on operational commands from external devices and transmits the camera's captured images or video via the wireless unit. This embodiment adds a camera imaging system with wireless connectivity to external devices within the intravesical floating drug delivery device. Through changes in the patient's body position, it allows direct observation of bladder tumors, potentially replacing traditional invasive cystoscopy and reducing the pain caused by cystoscopy for patients.

### Embodiment 6

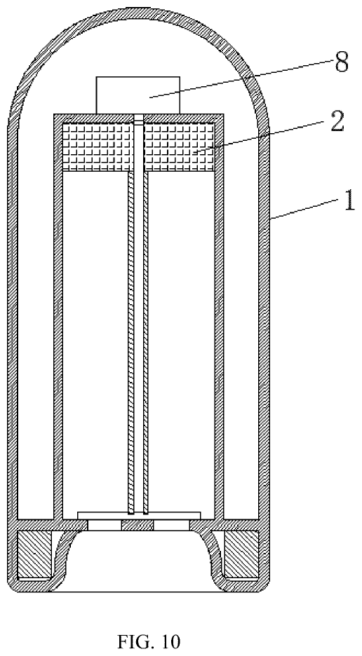

Compared to the previous embodiments, this embodiment involves a different layout design for the gas chamber based on the previous embodiments; the rest of the structural design is the same. As shown in FIG. 10, the gas chamber is configured as a semi-enclosed structure located at the head end and the side surfaces of the housing 1. Layers are provided at both the head end and the side surfaces of the housing 1 to form a semi-enclosed gas chamber. No layer is provided at the tail end drug outlet of the housing 1 to prevent insufficient buoyancy provided by the gas chamber within the limited size of the housing 1, while also achieving a more balanced mass distribution for the housing 1.

### Embodiment 7

This embodiment differs from Embodiments 1 and 3 in the composition and control principle of the control module 2. It also streamlines the structure by removing components related to the sensing electrodes 14; the rest of the structural design is the same. The control module 2 in this embodiment includes a storage unit and a processing unit. The storage unit is configured to store preset control signals or programs. These control signals include start time, interval time, baffle plate rotation angle, first electromagnet on/off signals, etc.

The processing unit is configured to read and process these control signals or programs, and control the operation of the drive module according to the signals or programs. When the start time arrives, the processing unit controls the drive module to perform the corresponding action. When the interval time arrives, the processing unit continues to control the drive module to perform the next corresponding action, such as rotating the baffle plate 10, energizing/de-energizing the first electromagnet, etc.

Compared to the interactive and real-time control scheme with external devices in Embodiments 1 and 3, this intravesical floating drug delivery device operates as a closed-loop system. Its control logic is preset according to medical advice or recommended usage and dosage before insertion into the bladder. The intravesical floating drug delivery device performs timed and quantified intravesical drug administration according to the preset logic inside the bladder and is simply removed upon completion.

To facilitate the insertion of the aforementioned intravesical floating drug delivery device into the bladder and its removal from the bladder, the present invention also provides a placement rod for the intravesical floating drug delivery device. This placement rod is detachably connectable to the intravesical floating drug delivery device. The diameter of the placement rod must comply with the insertion range of the urethra. After inserting the intravesical floating drug delivery device into the bladder via the urethra, it can disengage from the intravesical floating drug delivery device and be withdrawn from the urethra. When the intravesical floating drug delivery device needs to be removed, the placement rod can be inserted into the bladder, secured to the intravesical floating drug delivery device, and then used to extract the intravesical floating drug delivery device through the urethra. This placement rod can be implemented in various forms, as described in the following embodiments.

### Embodiment 8

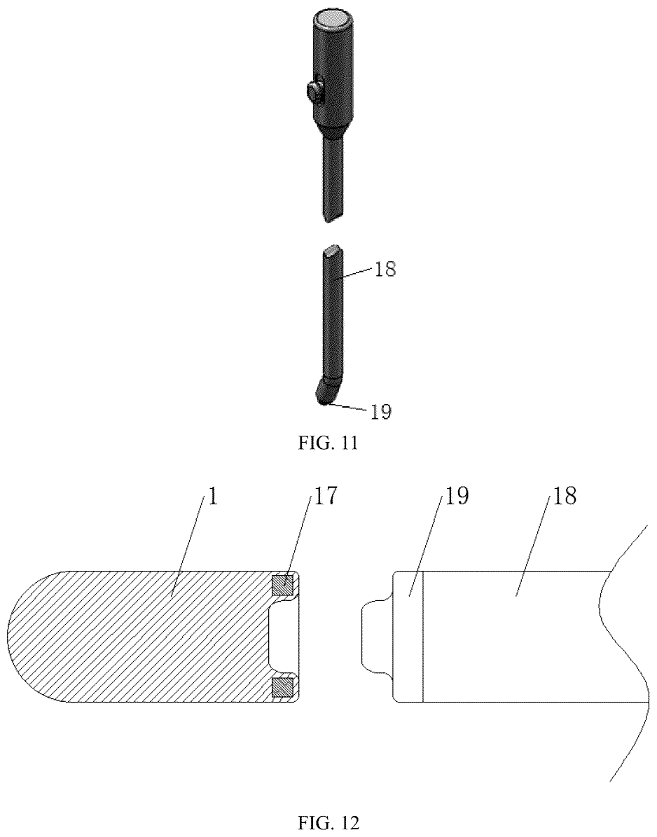

Please refer to FIGS. 11 and 12, This placement rod includes a rod body 18 and a second electromagnet 19. One end of the rod body 18 is provided with a docking head (which can be a cylindrical protrusion) matching the docking slot at the tail end of the housing 1. The purpose of the docking head and docking slot is to provide guidance, ensuring the end of the rod body 18 aligns correctly with the tail end of the housing 1, facilitating the subsequent magnetic attraction of the second electromagnet 19. The second electromagnet 19 is disposed on the docking head. The end of the rod body 18 away from the docking head is provided with a corresponding power source and switch. The switch controls the activation and deactivation of the second electromagnet 19.

When inserting the intravesical floating drug delivery device, the second electromagnet 19 is first activated. It generates a magnetic force that attracts the annular magnet 17 in the docking slot, thereby fixing the intravesical floating drug delivery device relative to the end of the rod body 18, facilitating the insertion of the intravesical floating drug delivery device into the bladder using the placement rod. After deactivating the second electromagnet 19, the intravesical floating drug delivery device naturally detaches from the placement rod, which is then withdrawn from the urethra. Similarly, when removing the intravesical floating drug delivery device, the placement rod is inserted into the bladder, the second electromagnet 19 is activated, and through the guidance of the docking head and docking slot, the second electromagnet 19 and the annular magnet 17 align and firmly attract, allowing the placement rod to extract the intravesical floating drug delivery device through the urethra.

### Embodiment 9

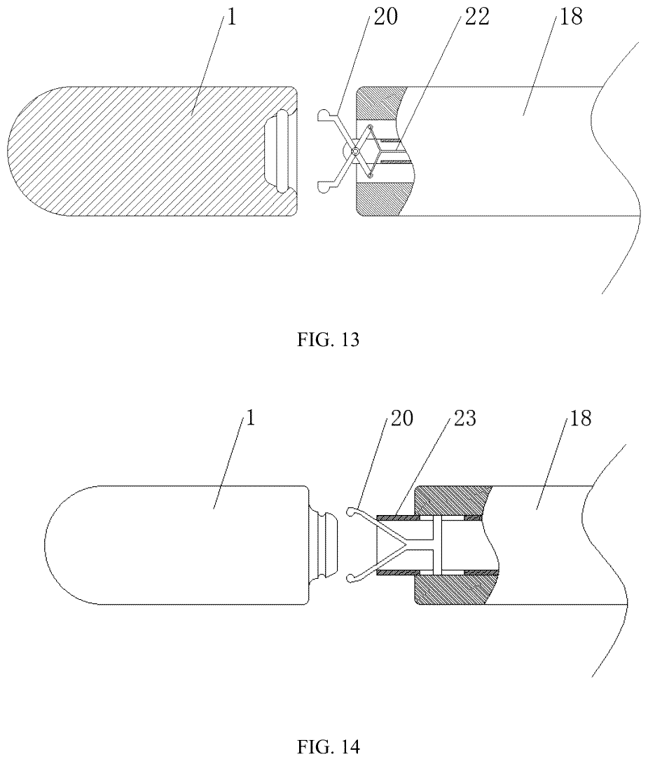

Please refer to FIG. 13, The placement rod in this embodiment includes a rod body 18, a clamp 20 installed at one end of the rod body 18, and an operating handle (not shown in the figure) at the other end. The clamp 20 is of a scissor-linkage type, similar to a scissors structure, and is mounted on the end of the rod body 18 via a connecting piece. A spring is also connected to the clamp 20. The function of this spring is to keep the clamp 20 in a normally open state. The operating handle is connected to one end of the clamp 20 via a pull cord 22. The operating handle is configured to drive the opening and closing of the clamp 20. Pulling the pull cord 22 causes the clamp 20 to close.

This clamp 20 is also used in conjunction with the docking slot on the housing 1. The docking slot can be provided with corresponding anti-slip grooves. When the placement rod needs to be fixed to the housing 1, the clamp 20 is inserted into the docking slot, and the operating handle is released. The clamp 20 opens outward under the action of the spring, thereby engaging the inner wall of the docking slot. The clamp 20 fixes the housing 1 through internal expansion. When the placement rod needs to be detached from the housing 1, the operating handle is used to pull the pull cord 22, controlling the clamp 20 to close. At this point, the clamp 20 can be disengaged from the docking slot, achieving separation between the clamp 20 and the housing 1.

### Embodiment 10

Please refer to FIG. 14, The placement rod in this embodiment includes a rod body 18, a clamp 20, and a limiting sleeve 23. The clamp 20 is made of elastic material, and its non-clamping end is fixed to the end of the rod body 18. The limiting sleeve 23 is slidably disposed within the inner wall of the rod body 18. The end of the rod body 18 away from the clamp 20 can be provided with an operating structure, such as a button, to control the sliding of the limiting sleeve 23.

The limiting sleeve 23 sleeves the clamp 20, allowing its end to abut against the back of the clamp 20. The degree of opening and closing of the clamp 20 is controlled by the extent to which the limiting sleeve 23 compresses it. For this purpose, a corresponding clamping portion can be provided at the tail end of the housing 1. The clamp 20 closes to grip the clamping portion, achieving fixation between the rod body 18 and the intravesical floating drug delivery device. Alternatively, the clamping portion may not be provided, and the clamp 20 can be inserted directly into the docking slot mentioned in the previous embodiments for fixation through internal expansion.

### Embodiment 11

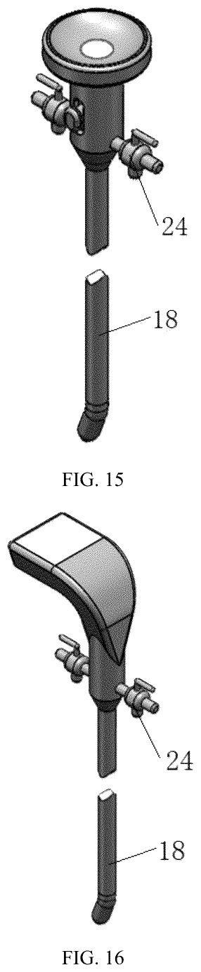

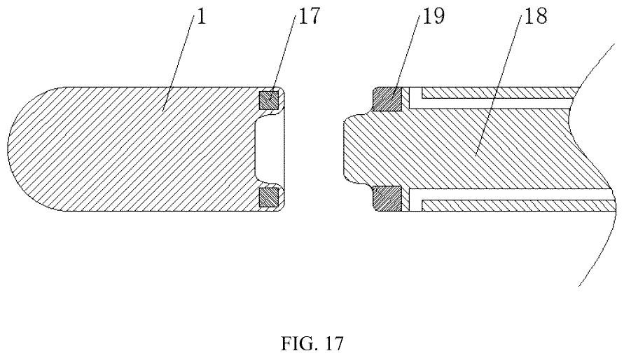

Compared to Embodiments 8, 9, and 10, this embodiment adds an additional endoscope system to the rod body 18 based on the previous embodiments. This endoscope system can be a traditional optical system, as shown in FIG. 15 (the optical eyepiece is located above the rod body 18), or an electronic system. The electronic system typically includes a CCD or CMOS lens. The lens captures the front image, then converts the image signal into an electrical signal. The electrical signal is transmitted along wires to a display screen. Finally, the electrical signal is processed by the image processing system within the display, forming a clear image on the screen for real-time viewing, as shown in FIG. 16 (the display screen is located above the rod body 18, used to display the image captured by the electronic objective lens for the operator to view).

Regardless of the system, its objective lens is installed at the end of the rod body 18 where the second electromagnet 19 or clamp 20 is located, allowing inspection of the bladder condition after the rod body 18 is inserted with the floating device. Care must be taken not to obstruct the lens's field of view. It is important to note that to avoid blocking the endoscope system's view, the cross-sectional area of the rod body 18 end should be slightly larger than that of the floating device. For example, if the rod body 18 end has a circular cross-section, the docking head and second electromagnet 19 are placed at an eccentric position on the connecting end face, leaving the remaining space for the lens.

Furthermore, for the clamp structures in Embodiments 9 and 10, the endoscope system also requires a design that avoids obstruction. For instance, if the clamp 20 is positioned in the center of the rod body 18 end face, the endoscope lens needs to be placed eccentrically; if the clamp 20 is positioned eccentrically on the rod body 18 end face, the endoscope lens can be placed in the center of the rod body 18 end face. Regardless of the layout, the goal is to prevent obstruction of the lens.

### Embodiment 12

Compared to Embodiments 8, 9, 10, and 11, this embodiment adds additional inlet and outlet pipelines to the rod body 18 based on the previous embodiments. Please refer to FIG. 17, one end opening of the inlet and outlet pipelines is located at the end of the rod body 18 near the second electromagnet 19, and the other end opening is located at the end of the rod body 18 away from the second electromagnet 19. The rod body 18 is also provided with corresponding connectors and control valves 24, connecting the inlet and outlet pipelines to an external water supply device. When the end of the placement rod near the second electromagnet 19 is inserted into the bladder, urine can be injected into and discharged from the bladder through the inlet and outlet pipelines, enabling cyclic irrigation or cooling of the bladder interior.

Any one of the intravesical floating drug delivery devices from Embodiments 1 to 7 can be combined with any one of the placement rods from Embodiments 8 to 12. Their combination forms a intravesical floating drug delivery device assembly. Through coordinated use, they enable the entire process of inserting the intravesical floating drug delivery device, releasing drugs, and removing it, effectively reducing patient suffering during bladder treatment.

The present invention also provides control methods corresponding to the intravesical floating drug delivery device. These control methods include two control principles, as described in the following embodiments.

### Embodiment 13

The control method in this embodiment is applied to the intravesical floating drug delivery devices described in Embodiments 1 to 6. The control method includes the following step: remotely controlling the drug release timing and/or drug release amount of the intravesical floating drug delivery device in the bladder in real-time based on the user's treatment condition or the presence of adverse reactions.

Taking the intravesical floating drug delivery devices from Embodiments 1, 2, and 3 as control objects, the drug release timing can be controlled in real-time by external devices (such as mobile phones or tablets) in signal connection with the intravesical floating drug delivery device. Alternatively, the drug release timing can be set, adjusted, etc., via the external devices. The drug release amount is achieved by controlling the number of drug chambers 4 opened, i.e., each drug chamber 4 contains one dose. If the therapeutic effect is insufficient, multiple chambers can be opened within the same time period to release multiple doses. If the therapeutic effect is significant and requires dose reduction, chambers can be left unopened during that period, or fewer chambers can be opened, thereby reducing the drug release amount. In this control method, real-time remote control and information reception from the inserted intravesical floating drug delivery device are possible, allowing for precise real-time control of dosage and timing based on disease progression or adverse reactions, maximizing therapeutic efficacy.

### Embodiment 14

The control method in this embodiment is applied to the intravesical floating drug delivery device described in Embodiment 7. The control method includes the following steps: (1) starting the drive module when a preset start time on the control module 2 arrives; (2) after the drive module is started, controlling the periodic operation of the drive module according to a preset cycle period on the control module 2: the cycle period includes a time t1 and a time t2; stopping the drive module when time t1 is reached; starting the drive module when time t2 is reached. Of course, it can also include the following steps: (3) monitoring the conduction signal generated by the sensing electrodes 14 and confirming the drug release status based on the conduction signal.

Using the structure from Embodiment 2 as an example, the start time is the time for the first activation of the drive module, set according to the actual situation. After the intravesical floating drug delivery device is inserted into the bladder and the start time is reached, the baffle plate 10 rotates. Its second through hole 21 rotates from the initial position (where the second through hole 21 is below the power supply module 3) to align with a first through hole 7, opening the corresponding drug chamber 4 and releasing the drug into the bladder. Time t1 can be set to 1-3 seconds. After the drive module starts, it stops within 1-3 seconds upon reaching the next position, i.e., the second through hole 21 rotates from its current position to align with the next first through hole 7, opening the corresponding drug chamber 4. Time t2 is the drug interval, generally set to 3-6 weeks. When time t2 is reached, the drive module restarts, and subsequent cycles repeat the t1 and t2 time actions.

If using the structure from Embodiment 3 as the control object, simply change the control object from the baffle plate 10 to the first electromagnet 11. Activate different first electromagnets 11 within the cycle period to push all the drugs from the corresponding drug chamber 4 into the bladder. If using the structure from Embodiment 4 as the control object, the action mode differs slightly: time t1 is the energization time of the first electromagnet 11, controlling the amount of drug expelled from the drug chamber 4 by the duration of the repulsive force.

For example, if the entire drug in the drug chamber 4 can be expelled in 5 seconds (equivalent to the first electromagnet 11 acting on the permanent magnet 12 for 5 seconds), time t1 can be set to 1 second. That is, the first electromagnet 11 is energized for 1 second, applying a 1-second push to the permanent magnet 12, thereby expelling one-fifth of the drug amount (as the overall size of the intravesical floating drug delivery device is small, the decrease in repulsive force when the permanent magnet 12 moves away from the first electromagnet 11 can be considered negligible, or different t1 times can be set).

In this control method, the intravesical floating drug delivery device operates as a closed-loop system. Its control method is programmed and burned into the integrated circuit of the control module 2, with relevant parameters set according to medical advice or recommended usage and dosage before insertion into the bladder. The intravesical floating drug delivery device performs timed and quantified intravesical drug administration inside the bladder according to the preset control method. This intravesical floating drug delivery device is a disposable item. Continuous cyclic operation according to the above method does not affect drug release or the bladder environment until the power is depleted or the device is removed from the bladder.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A wireless intelligently controlled drug-releasing intravesical floating drug delivery device, comprising:
a housing configured to enter the bladder via the urethra and float within the intravesical urine, the housing is internally provided with at least one drug chamber for containing drug and a gas chamber for providing buoyancy to float the entire intravesical floating drug delivery device within the intravesical urine;
a drive module disposed within the housing; the drive module is configured to cause drug to be discharged from the drug chamber to the exterior of the housing in a timed and/or quantified manner;
a control module configured to control the operation of the drive module; and
a power supply module disposed within the housing; the power supply module is configured to supply power to the drive module.

2. The device according to claim 1, wherein the housing is connected with a plurality of partition plates; the plurality of partition plates partition the interior of the housing into a plurality of drug chambers; the cross-section of each of the drug chambers is fan-shaped and all cross-sections are concentrically arranged; the housing is provided with first through holes corresponding one-to-one with the plurality of drug chambers, the first through holes are configured for the ingress and egress of corresponding drug.

3. The device according to claim 2, wherein a central tube is disposed at the center of the plurality of fan-shaped chambers; the drive module comprises:
a motor configured to be controlled by the control module and rotate by a set angle;
a transmission rod rotatably disposed inside the central tube; one end of the transmission rod is connected to an output shaft of the motor; and
a baffle plate connected to the other end of the transmission rod, wherein the baffle plate is rotatably connected to the housing and configured to seal the side of the housing provided with the first through holes; the baffle plate is configured to close or open the first through holes upon rotation.

4. The device according to claim 1, wherein the drive module comprises:
a first electromagnet disposed at one end of the drug chamber; the first electromagnet is configured to be controlled by the control module to generate a magnetic force;
a permanent magnet sealed and slidably disposed along an inner wall of the drug chamber; the magnetic force of the permanent magnet is repulsive to the magnetic force of the first electromagnet, and a portion of the drug chamber on a side of the permanent magnet away from the first electromagnet is a drug reservoir; and
a check valve; the housing is provided with a first through hole in communication with the drug reservoir, the check valve is disposed within the first through hole, and an opening direction of the check valve is from the interior to the exterior of the housing.

5. The device according to claim 1, wherein two adjacent sensing electrodes are disposed within each drug chamber; the two sensing electrodes are configured to generate a conduction signal after contacting the urine or drug within the bladder, the conduction signal is transmitted to an external device via the control module.

6. The device according to claim 1, wherein the control module comprises:
a storage unit configured to store preset control programs; and
a processing unit configured to control the operation of the drive module according to the control programs.

7. The device according to claim 1, wherein the control module comprises:
a wireless unit configured to remotely receive signals from outside the housing; and
a processing unit configured to control the operation of the drive module based on the signals.

8. The device according to claim 7, wherein the device further comprises:
a camera module disposed within the housing; the camera module is configured to capture images of the bladder inner wall through the housing and transmit the captured video to the exterior via the wireless unit;
a light source disposed within the housing; the light source is configured to illuminate the shooting direction of the camera module through the housing; the power supply module is configured to supply power to the camera module and the light source.

9. The device according to claim 1, wherein the gas chamber is configured as a semi-enclosed structure located at the head end and the side surfaces of the housing.

10. The device according to claim 1, wherein the housing has a diameter not greater than 8 mm and an overall length not greater than 4 cm, and the outer surface of the housing is coated with an antibacterial, hydrophilic, anti-adhesion coating.

11. The device according to claim 4, wherein the check valve comprises a rigid film provided with an incision, and when subjected to pressure from the drug within each drug chamber, the rigid film opens to allow unidirectional outflow of the drug.

12. A placement rod for a intravesical floating drug delivery device, comprising a rod body and an engagement structure at one end of the rod body, wherein the engagement structure is configured to detachably engage with the intravesical floating drug delivery device according to claim 1.

13. The placement rod according to claim 12, wherein the housing is externally provided with a docking slot, and a magnet is connected within the docking slot; the engagement structure comprises a docking head matching the docking slot and a second electromagnet disposed on the docking head, the second electromagnet is configured to attract the magnet when energized.

14. The placement rod according to claim 12, wherein the engagement structure comprises an operating handle and a clamp for securing the intravesical floating drug delivery device; the clamp is disposed at one end of the rod body, the operating handle is disposed at the other end of the rod body; the operating handle is configured to drive the opening and closing of the clamp.

15. The placement rod according to claim 12, wherein the placement rod is integrated with an endoscope system, the endoscope system is an optical or electronic system.

16. The placement rod according to claim 12, wherein the placement rod is provided with an inlet pipeline and an outlet pipeline; one end opening of the inlet pipeline and outlet pipeline is located at the end of the placement rod, and the other end is connected to an external water supply device for injecting and discharging urine into and from the bladder.

17. A intravesical floating drug delivery device component, comprising the intravesical floating drug delivery device according to claim 1 and the placement rod according to claim 12.

18. A control method for a wireless intelligently controlled drug-releasing intravesical floating drug delivery device, comprising the following step:
remotely transmitting a control signal to a control module of the intravesical floating drug delivery device to adjust a drug release timing or a drug release amount.

19. A control method for a wireless intelligently controlled drug-releasing intravesical floating drug delivery device having a control module and a drive module, comprising the following step:
starting the drive module when a preset start time on the control module arrives; and
after the drive module is started, controlling the periodic operation of the drive module according to a preset cycle period, wherein the cycle period comprises an operating time t1 and an interval time t2; stopping the drive module when the operating time t1 is reached; and starting the drive module when the interval time t2 is reached.

20. The control method according to claim 19, further comprises the following step:
monitoring the conduction signal generated by the sensing electrodes and confirming the drug release status based on the conduction signal.
